(19) United States
(12) Patent Application Publication
(10) Pub. No.: US 2006/0015102 A1
(43) Pub. Date: Jan. 19, 2006

(54) Fastener implant for osteosynthesis of fragments of a first metatarsal bone that is broken or osteotomized in its proximal portion and a corresponding osteosynthesis method

(76) Inventors: Eric Rene Marie Toullec, Bruges (FR); Jerome Alain, Limoges (FR); Christophe Jean-Michel Nicolas Camelot, Vincennes (FR); Wilfrid Olivier Graff, Paris (FR)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/173,589

(22) Filed: Jul. 1, 2005

(30) Foreign Application Priority Data
Jul. 15, 2004 (FR) FR-04 07891

(51) Int. Cl. A61F 2/30

(52) U.S. Cl. 606/69

(57) ABSTRACT

The invention provides a fastener implant for osteosynthesis of fragments of the first metatarsal bone in its proximal portion situated towards the tarsal bone, the implant comprising at least a fastener element for fastening via a fastener face on or against the outside surface of the first metatarsal bone in order to hold the bone fragments together, wherein said fastener face includes at least one anatomical surface portion of shape that is substantially complementary to the shape of the plantar surface of the proximal portion of the first metatarsal bone.

### FIELD OF THE INVENTION

The present invention relates to the general technical field of fastener implants for ensuring osteosynthesis of bone fragments, and in particular two fragments of a bone that has broken or has been sectioned by osteotomy.

The present invention relates more particularly to the technical field of devices and methods for treating certain pathological conditions of the metatarsus such as fractures or deformations of the big toe, in particular those known under the scientific term “hallux valgus”.

The present invention relates to a fastener implant for osteosynthesis of fragments of the first metatarsal bone in its proximal portion, situated towards the tarsal bone, the implant comprising at least one fastener element for being fastened via a fastener plate on or against the outside surface of the first metatarsal bone, for example, and essentially on either side of the zone of separation between the bone fragments, in order to hold them together.

The present invention also relates to a method of osteosynthesis of fragments of the first metatarsal bone in its proximal portion, situated towards the tarsal bone.

### BACKGROUND OF THE INVENTION

Several techniques and several associated devices exist for correcting deformations of the “hallux valgus” type.

“Hallux valgus” is a deformation of the metatarsus constituting exaggerated lateral deflection of the big toe. Once the deformation is initiated, it generally becomes more accentuated with increasing metatarsal-phalanx angle. This deformation is often accompanied by painful swelling commonly known as a “bunion”.

Numerous surgical methods have been proposed for correcting deformations of the “hallux valgus” type. Two main categories of operation are presently in use, firstly so-called distal osteotomies which consist in sectioning the first metatarsal bone in its distal portion (or metatarsal head) situated towards the phalanx, and secondly so-called proximal osteotomies which consist in sectioning the first metatarsal bone in its proximal portion (or metatarsal base) situated towards the tarsal bone.

The first category of operations generally gives good results for small angles of deflection between the first and second metatarsal bones. However, this category of intervention becomes much less satisfactory for large angles of deflection, and in particular when the angle between the first and second metatarsal bones exceeds 10° or 15°. Under such circumstances, the second category of operations is preferred. Proximal osteotomy thus consists in sectioning the first metatarsal bone, e.g. using a lateral approach, to constitute at least two bone fragments in its proximal portion situated towards the tarsal bone. Thereafter the bone fragments are realigned so as to correct the angular deflection. Osteosynthesis of the bone fragments is then ensured by using a fastener system, e.g. formed by screws or even a plate positioned on the dorsal or lateral portion of the first metatarsal bone.

Known fastener systems nevertheless present several non-negligible drawbacks, both in terms of being difficult to put into place and in terms of mechanical strength and ability to hold the bone fragments together effectively.

In the event of excessive compression, screw fastener systems can make the bone friable, which can lead to the bone fragments separating.

From this point of view, plate systems give better results, but they too can lead to problems, in particular while the bone is consolidating. During this period, the bone can be subjected, in particular during walking, to mechanical stresses that are liable to cause the bone fragments to separate.

When performing a “hallux valgus” operation, it is generally desirable for the patient to be able to use the foot again as quickly as possible. The foot that has been operated on is thus quickly returned to pressing against the ground while the consolidation stage is still taking place, and as a result, during walking and under the action of the weight of the patient's body, this can lead to torque tending to load the fastener system very heavily and to separate the bone fragments. This phenomenon is generally accentuated when fastener plates are placed on the dorsal or lateral surface of the proximal portion of the first metatarsal bone. Dorsal fastener plates work in bending and can find it difficult to oppose the phenomenon of the bone opening on the plantar side, even though it is specifically on the plantar side that the bone tends to open up more during walking.

Furthermore, it can happen that fastener plates are poorly positioned, in particular because they are often difficult to put into place. Such poor positioning can not only be harmful to the stability of the implant, but under the effect of repeated mechanical stresses, it can also lead to the implant progressively loosening or even being torn off.

The positioning of the fastener implant therefore turns out to be a fundamental parameter for ensuring that the implant is as insensitive as possible to external mechanical stresses, particularly those associated with walking, and that it continues to hold the bone fragments together properly.

### OBJECTS AND SUMMARY OF THE INVENTION

The objects assigned to the invention consequently seek to propose a novel fastener implant that does not present the drawbacks listed above and that enables fractures to be repaired or deformations to be corrected, in particular deformations of the most severe “hallux valgus” type, while still allowing the patient to return quickly to daily physical activities, such as walking.

Another object of the invention seeks to propose a novel fastener implant having greater ability to withstand mechanical stress than prior devices, in particular during the immediate postoperative period.

Another object of the invention is to propose a novel implant of stability that is not degraded by repeated stresses, thus avoiding separation of the bone fragments during consolidation.

Another object of the invention seeks to propose a novel fastener implant of position that is locked, such that the implant cannot slide while it is under stress, in particular during physical activities such as walking.

Another object of the invention is to propose a novel fastener implant which is particularly easy to put into place and position properly on the proximal plantar surface of the first metatarsal bone.

Another object of the invention is to propose a novel fastener implant which can be put into place blind.

Another object of the invention is to propose a novel fastener implant that is compact, in particular relative to the nearby soft tissue.

Another object of the invention is to propose a novel fastener implant that enables bone fragments to be properly compressed, without weakening them.

Another object of the invention is to propose a novel fastener implant that is atraumatic.

The objects given to the invention are achieved with the help of a fastener implant for osteosynthesis of fragments of the first metatarsal bone in its proximal portion situated towards the tarsal bone, the implant comprising at least a fastener element for fastening via a fastener face on or against the outside surface of the first metatarsal bone in order to hold the bone fragments together, wherein said fastener face includes at least one anatomical surface portion of shape that is substantially complementary to the shape of the plantar surface of the proximal portion of the first metatarsal bone.

The objects given to the invention are also achieved with the help of a method of performing osteosynthesis on fragments of a first metatarsal bone in its proximal portion situated towards the tarsal bone, the method comprising a fastening step of fastening an implant provided with a fastener element to the outside surface of the first metatarsal bone, during which step the fastener element is positioned via a plantar approach against the proximal plantar surface of the first metatarsal bone, in such a manner that said fastener element extends on either side of the separation zone between the fragments.

### BRIEF DESCRIPTION OF THE DRAWINGS

Other objects and advantages of the invention appear more clearly on reading the following description, and with the help of the accompanying drawings that are provided merely by way of non-limiting illustration, in which:

FIG. 1 is a diagrammatic view from beneath of the skeleton of a human left foot on which an osteotomy has been performed on the proximal portion of the first metatarsal bone;

FIG. 2 is a view from beneath of the skeleton of a human metatarsus presenting deformation of the “hallux valgus” type on the first metatarsal bone, before operating;

FIG. 3 is a side view in elevation outlining the skeleton of a human right foot having a fastener implant of the invention secured thereto;

FIGS. 4 and 5 are side views of a fastener element in accordance with the invention;

FIGS. 6a and 6b are plan views of left and right fastener elements designed specifically for a left foot and for a right foot; and

FIG. 7 is a plan view of a preferred embodiment of the fastener element in accordance with the invention.

### MORE DETAILED DESCRIPTION

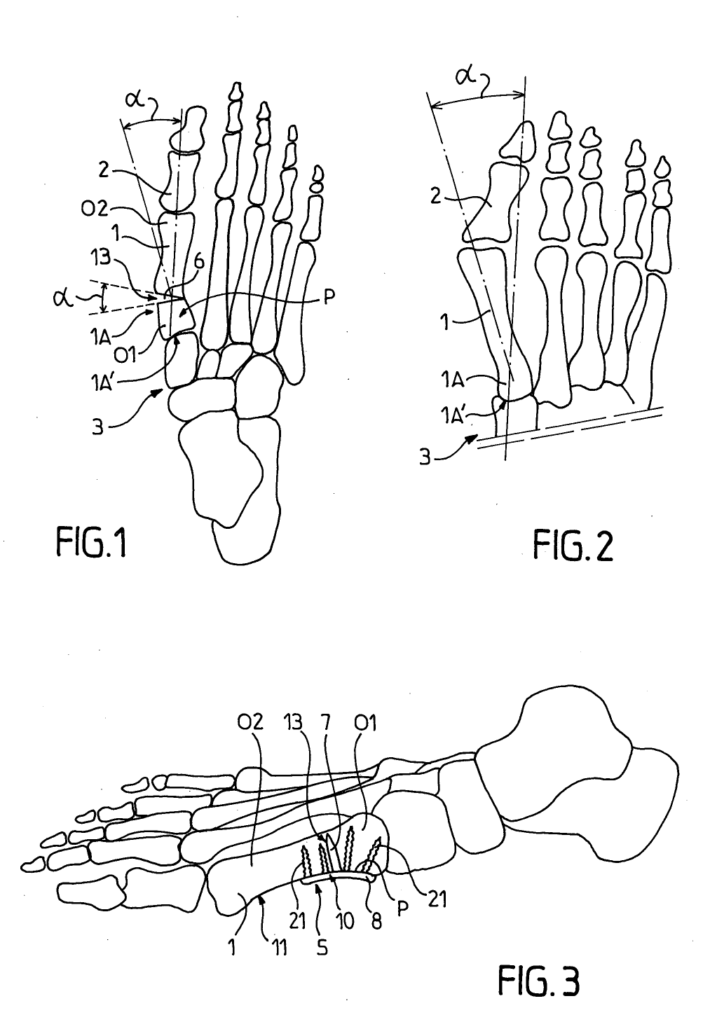

FIG. 1 shows the skeleton of a human foot, and in the figure there can be seen the first metatarsal bone 1 between the first phalanx 2 of the big toe and the tarsal bone 3. As shown in FIG. 1, the first metatarsal bone 1 has been sectioned by a lateral approach in its proximal portion 1A situated towards the tarsal bone 3, e.g. for the purpose of correcting deformation of the “hallux valgus” type, as shown in FIG. 2, which corresponds to an abnormal angle of deflection α of the first metatarsal bone.

In the description below, it is assumed that the first metatarsal bone has been subjected to osteotomy in its proximal portion 1A, e.g. subdividing it into two bone fragments O1 and O2. Under such circumstances, the fastener implant in accordance with the invention is intended to ensure osteosynthesis of the two bone fragments O1 and O2 after the osteotomy has been performed. Nevertheless, the fastener implant of the invention is not restricted to this type of application and it could equally well be used for repairing fractures of the proximal portion of the first metatarsal bone, without thereby going beyond the ambit of the invention.

FIG. 3 shows a fastener implant 5 fastened to the first metatarsal bone 1 in order to ensure osteosynthesis between the bone fragments O1 and O2, following the osteotomy as shown in FIG. 1. In order to fill the gap 6 separating the two bone fragments O1 and O2, a wedge 7 (or spacer) is placed between the two bone fragments O1, O2.

In the meaning of the invention, the bone fragments O1 and O2 are not necessarily disjoint, i.e. the first metatarsal bone 1 is not necessarily sectioned or fractioned across its entire diameter, and there may remain a zone of connection between the bone fragments O1 and O2. The fastener implant 5 is thus intended to ensure osteosynthesis between at least two bone fragments O1 and O2, and possibly between some larger number of bone fragments.

In the invention, the fastener implant 5 comprises at least one fastener element 8 that is preferably in the form of a plate 9. The fastener element 8, and more precisely, the plate 9, has a fastener face 10 via which the fastener element 8 is to be fastened on or against the bony outside surface 11 of the first metatarsal bone 1. The fastener face 10 thus forms the “inside” face of the fastener element 8. The fastener element 8 also advantageously includes an outside face 12 substantially opposite to the fastener face 10.

The bone fragments O1 and O2 are separated by a separation zone 13, and the fastener element 8 is for fastening on either side thereof in order to ensure that the bone fragments O1 and O2 are held together and osteosynthesis takes place between them.

According to an essential characteristic of the invention, the fastener face 10 includes at least one anatomical surface portion 14 of shape that is substantially complementary to the shape of the plantar surface P of the proximal portion 1A of the first metatarsal bone 1.

In particularly advantageous manner, the anatomical surface portion 14 is shaped, i.e. it is specifically designed and dimensioned, so as to fit substantially snugly over the plantar surface P of the proximal portion 1A of the first metatarsal bone 1.

The term “planar surface” refers to the bony surface situated beneath the proximal portion 1A of the first metatarsal bone 1, i.e. in distinction to the dorsal and lateral bony surfaces of the first metatarsal bone.

By fitting anatomically to the proximal plantar surface P of the first metatarsal bone 1, and by pressing intimately against the bony surface, the fastener element 8 provides improved resistance to mechanical stress, in particular stress associated with walking. The geometrical shape of the fastener element 8 also ensures better positioning and better pressing of the implant against the bone fragments to be united, thus making it possible to obtain results that are better from the clinical point of view.

In addition, the special shape of the fastener element 8 enables it to be put into place very quickly, and blind, since it naturally takes up its position in the desired location. The fastener element 8 is thus preshaped during fabrication, i.e. long before the surgical operation, so as to match the shape of the proximal plantar surface P of the first metatarsal bone 1. The present invention thus makes it possible to anticipate the positioning of the fastener implant 5 and to make it immediately operational and functional.

In particularly advantageous manner, the anatomical surface portion 14 of the fastener element 8 is substantially convex so as to fit the concave shape of the proximal plantar surface P of the first metatarsal bone 1. The fastener element 8 thus advantageously follows the curvature of the proximal plantar surface P of the first metatarsal bone 1, thereby giving it very great mechanical stability.

Advantageously, the anatomical surface portion 14 includes at least a “proximal” surface 15 situated towards the proximal end 1A′ of the first metatarsal bone 1. The proximal surface 15 is advantageously inclined at a predetermined angle relative to the remaining fraction of the anatomical surface portion 14.

In even more preferred manner, the anatomical surface portion 14 includes at least a “distal” surface 16, situated towards the end of the fastener element 8 that is remote from the proximal surface 15, and also an intermediate surface 17 situated between the proximal surface 15 and the distal surface 16, in such a manner that the proximal, intermediate, and distal surfaces 15, 16, and 17 are perceptibly inclined relative to one another.

The relative positions and orientations of these surfaces are of great importance since they determine the positioning and the orientation of the fastener implant 5. Thus, a poor three-dimensional configuration for these surfaces would have a negative effect on the stability of the implant and on its ability to withstand mechanical stresses.

In preferred manner, the proximal, intermediate, and distal surfaces 15, 16, and 17 are substantially plane.

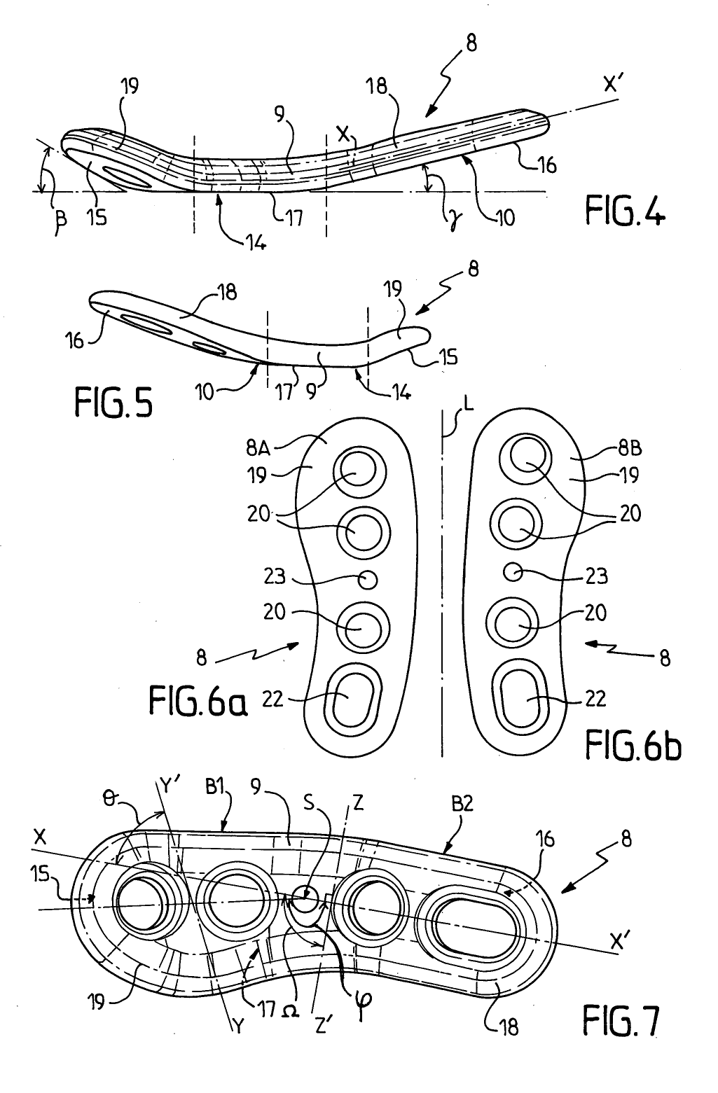

According to a particularly advantageous characteristic of the invention, the proximal and intermediate surfaces 15 and 17 are inclined relative to each other by a proximal bend angle β lying in the range 5° to 30°, and preferably of the order of 25° (FIG. 4). The proximal and intermediate surfaces 15 and 17 thus meet at a proximal bend axis Y-Y′ which, by way of illustration, coincides with an imaginary fold axis of the fastener element 8 on either side of which the proximal and intermediate surfaces 15 and 17 are inclined.

Advantageously, the distal and intermediate surfaces 16 and 17 are inclined relative to each other by a distal bend angle γ lying in the range 5° to 25°, and preferably of the order of 15°. The distal and intermediate surfaces 16 and 17 advantageously meet about a distal bend axis Z-Z′ that coincides substantially with an imaginary fold axis of the fastener element, on either side of which the distal and intermediate surfaces 16 and 17 are inclined.

Roughly, the fastener element 8 extends longitudinally along a longitudinal axis X-X′. This longitudinal axis X-X′ coincides substantially with the axis along which the distal surface 16 or the distal segment 18 of the fastener element 8 extends longitudinally.

The proximal bend axis Y-Y′ is advantageously inclined relative to the longitudinal axis X-X′ by an angle θ lying in the range 60° to 80°, and preferably of the order of 70°. The distal bend axis Z-Z′ is inclined relative to the longitudinal axis X-X′ by an angle Ω lying in the range 80° to 100°, and preferably of the order of 90°.

Advantageously, opposite from the distal segment 18, the fastener element 8 has a proximal segment 19 that is spatula-shaped, being perceptibly wider than the remainder of the fastener element 8, and in particular wider than the distal segment 18. By means of this spatula shape, contact between the fastener element 8 and the bony surface is significantly improved, thus enabling the mechanical stresses exerted on this portion of the fastener implant 5 to be better distributed. The implant can then bear in intimate and stable manner against the bony surface of the first metatarsal bone 1, thus being more effective in absorbing all of the mechanical stresses exerted on the bone fragments O1 and O2.

The spatula-shaped proximal segment 19 also constitutes means for distinguishing a left fastener element 8A for putting into place on the first metatarsal bone 1 of a left foot from a right fastener element 8B for putting into place on the first metatarsal bone of the right foot by sight or by touch. As shown in FIGS. 6A and 6B, the right and left fastener elements 8A and 8B are symmetrical about the sagittal plane L, i.e. about the vertical plane intersecting the body from the anterior side to the posterior side, thus subdividing the body into a right half and a left half.

As shown in FIG. 7, the fastener element 8 advantageously comprises at least two branches, i.e. a proximal branch B1 and a distal branch B2 disposed substantially in a V-configuration. Thus, if a plane that is substantially tangential to the distal surface 16 of the fastener element 8 is defined, then the branches B1 and B2 extend, in projection on said tangential plane, in such a manner as to form a V-configuration that is wide open. The angle φ at the apex S of the V-shape advantageously lies in the range 160° to 175°, and is preferably about 170°.

In order to minimize the size of the fastener implant 5, and in particular in order to limit the invasive and aggressive nature of this implant relative to the soft tissue surrounding the bone fragments, the outside face 12 of the plate 9 preferably extends substantially parallel to the fastener face 10. Thus the entire fastener element 8 fits closely to the shape of the first metatarsal bone 1 in its proximal zone 1A, and thus forms an anatomical fastener implant 5 reproducing the anatomical characteristics of the first metatarsal bone 1, so as to make the implant invisible to the soft tissue. In order to further improve the atraumatic nature of the fastener implant 5, the outside face 12 is preferably smooth with edges that are rounded or dulled.

In contrast, the fastener face 10 is preferably rough in appearance, e.g. due to sandblasting, so as to improve engagement and adhesion of the fastener face 10 against the bony surface of the first metatarsal bone 1 and prevent the implant from sliding or moving relative to the bone once the fastener element 8 has been put into place in its functional position.

Fastening the implant 5 to the bony surface of the first metatarsal bone 1 constitutes a crucial step in the surgery insofar as a poorly positioned implant, or even an implant that is not uniformly tightened against the bone, can lead to the bone fragments O1 and O2 progressively becoming separated. Under such circumstances, in order to improve fastening of the fastener implant 5 against the first metatarsal bone 1, the fastener element 8 advantageously includes a plurality of through openings 20 distributed at regular intervals along the length of the fastener element 8 and suitable for receiving anchor elements 21 of the anchor screw type.

In order to lock the position of the fastener element 8 on the first metatarsal bone 1, the anchor elements 21 preferably comprise at least one screw and one lock-screw mounted in association. The through openings 20 preferably have an inside thread enabling the lock-screw to be tightened until it comes to bear against the head of the screw.

Such screw and lock-screw fastener systems may be based on the Surfix® concept, for example, as is well known to the person skilled in the art and is therefore not described in detail herein.

In still more preferable manner, the fastener element 8 includes a through oblong opening 22 preferably in the distal segment 18 of the fastener element 8. This oblong opening 22 enables bone compression to be performed so as to move the bone fragments O1, O2 effectively towards each other.

Preferably, the fastener element 8 also includes a non-through indentation 23 that is preferably situated substantially in the middle portion of its outside face 12 and within which a clamping tool of the forceps type can bear in order to clamp the implant against the first metatarsal bone 1.

Before and during the positioning of the anchor elements 21, the use of clamping forceps can release at least one of the hands of the surgeon and thus make it possible to optimize positioning of the fastener element 8. By way of example, such forceps may have three clamping points, a first clamping point in the indentation 23 and two clamping points for coming into contact with each of the bone fragments O1 and O2.

The present invention also provides a method of treating pathological conditions of the metatarsus such as fractures or deformations of the big toe, and in particular it provides a method of osteosynthesis.

The present invention provides a method of performing osteosynthesis between fragments O1 and O2 of a first metatarsal bone 1 in its proximal portion 1A situated towards the tarsal bone, the method comprising a step of fastening an implant provided with a fastener element 8 to the outside surface 11 of the first metatarsal bone, during which step, the fastener element 8 is positioned against the proximal plantar surface P of the first metatarsal bone 1 via a plantar approach, in such a manner that said fastener element 8 extends on either side of the separation zone 13 between the fragments O1 and O2.

More precisely, the fastener face 10 of the fastener element 8 is positioned directly on or against the proximal plantar surface P of the first metatarsal bone 1.

Advantageously, prior to the fastening step, the method of the invention includes a preparation step during which an incision is made and an access path is opened from the arch of the foot towards the plantar surface P of the first metatarsal bone 1, so as to enable the fastener element 8 to be inserted.

When treating a “hallux valgus” type deformation of the big toe, osteotomy is advantageously performed during the preparation step on the proximal portion 1A of the first metatarsal bone 1 which is sectioned in such a manner as to form the bone fragments O1 and O2.

Since the fastener element 8 is of anatomical shape, the fastening step takes place directly without any step of preforming or preshaping the fastener element 8. Because of the specific shape of the fastener element 8, and in particular because of the convex shape of its fastener face 10, no intermediate step of preforming the fastener element 8 is needed in order to give it a shape that its compatible with the plantar surface P. The fastener element 8 thus presents its functional shape on being fabricated, thus sparing the surgeon a series of laborious folding operations on the fastener plate 9 in order to adapt it to the specific shape of the plantar surface P.

The surgical method in accordance with the invention thus constitutes a method that is essentially performed by a plantar approach.

The invention thus makes it possible to reduce considerably the time required for the surgical operation and also limits any risk of error in the operation.

Another advantage of the invention is that it enables the fastener implant to be put into place blind while taking only a minimum number of precautions.

The invention also makes it possible to reduce significantly the risk of slack after the fastener implant has been put into place on the bony surface of the first metatarsal bone, and thus make it possible to improve considerably both the mechanical strength of the bone fragments taken together, and also the quality of osteosynthesis.

What is claimed is:

1. A fastener implant for osteosynthesis of fragments of the first metatarsal bone in its proximal portion situated towards the tarsal bone, the implant comprising at least a fastener element for fastening via a fastener face on or against the outside surface of the first metatarsal bone in order to hold the bone fragments together, wherein said fastener face includes at least one anatomical surface portion of shape that is substantially complementary to the shape of the plantar surface of the proximal portion of the first metatarsal bone.

2. An implant according to claim 1, wherein the anatomical surface portion is shaped in such a manner as to fit substantially snugly against the plantar surface of the proximal portion of the first metatarsal bone.

3. An implant according to claim 1, wherein the anatomical surface portion is substantially convex so as to match the concave shape of the plantar surface of the proximal portion of the first metatarsal bone.

4. An implant according to claim 1, wherein the anatomical surface portion comprises at least a “proximal” surface situated towards the proximal end of the first metatarsal bone, said proximal surface being inclined relative to the remaining fraction of the anatomical surface portion.

5. An implant according to claim 4, wherein the anatomical surface portion includes at least a “distal” surface situated towards the end of the fastener element that is opposite from the proximal surface, and an intermediate surface situated between the proximal surface and the distal surface, said proximal, intermediate, and distal surfaces being perceptibly inclined relative to one another.

6. An implant according to claim 5, wherein the proximal, intermediate, and distal surfaces are substantially plane.

7. An implant according to claim 5, wherein the proximal and intermediate surfaces are inclined relative to each other by a proximal bend angle lying in the range 5° to 30°, and preferably of the order of 25°.

8. An implant according to claim 5, wherein the distal and intermediate surfaces are inclined relative to each other by a distal bend angle lying in the range 5° to 25°, and preferably of the order of 15°.

9. An implant according to claim 5, wherein the fastener element extends longitudinally along a longitudinal axis, and the proximal and intermediate surfaces are united via a proximal bend axis, said proximal bend axis being inclined relative to the longitudinal axis at an angle lying in the range 60° to 80°, and preferably of the order of 70°.

10. An implant according to claim 5, wherein the fastener element extends longitudinally along a longitudinal axis, and the distal and intermediate surfaces are united via a distal bend axis, said distal end axis being inclined relative to the longitudinal axis at angle lying in the range 80° to 100°, and preferably of the order of 90°.

11. An implant according to claim 1, wherein the fastener element includes a proximal segment of spatula shape, that is significantly wider than the remainder of the fastener element.

12. An implant according to claim 1, wherein the fastener element comprises at least two branches, namely a proximal branch and a distal branch that are disposed substantially in a V-configuration.

13. An implant according to claim 12, wherein the angle at the apex of the V-configuration lies in the range 160° to 175°, and is preferably of the order of 170°.

14. An implant according to claim 1, wherein the fastener element is formed by a plate provided with an outside face opposite from the fastener face, said outside face extending substantially parallel to the fastener face.

15. An implant according to claim 14, wherein said outside face is substantially smooth.

16. An implant according to claim 1, wherein the fastener face presents a rough appearance.

17. An implant according to claim 1, wherein the fastener element includes a plurality of through orifices distributed around its length and adapted to receive anchor elements of the anchor screw type.

18. An implant according to claim 17, wherein the anchor elements comprise at least one screw and one lock-screw, mounted in association in such a manner as to lock the fastener element in position.

19. An implant according to claim 1, wherein the fastener element includes a substantially oblong through opening enabling the bone fragments to be moved towards each other.

20. An implant according to claim 1, wherein the fastener element includes an indentation preferably situated substantially in the middle portion of its outside face and suitable for receiving a clamping tool of the forceps type so that it can clamp the fastener implant against the first metatarsal bone.

21. An implant according to claim 1, wherein the outside face of the fastener element has rounded edges.

22. A method of performing osteosynthesis on fragments of a first metatarsal bone in its proximal portion situated towards the tarsal bone, the method comprising a fastening step of fastening an implant provided with a fastener element to the outside surface of the first metatarsal bone, during which step the fastener element is positioned via a plantar approach against the proximal plantar surface of the first metatarsal bone, in such a manner that said fastener element extends on either side of the separation zone between the fragments.

23. A method according to claim 22, including, prior to the fastening step, a preparation step during which an incision is made and an access path is prepared from the arch of the foot towards the plantar surface of the first metatarsal bone.

24. A method according to claim 23, wherein, during the preparation step, osteotomy is performed on the proximal portion of the first metatarsal bone, sectioning it in such a manner as to form the fragments.

25. A method according to claim 22, wherein the fastener element presents an anatomical shape and the fastening step is performed directly, without any step of preforming the fastener element.
